(19) United States
(12) Patent Application Publication
(10) Pub. No.: US 2015/0202088 A1
(43) Pub. Date: Jul. 23, 2015

(54) All-Weather Goggle

(71) Applicant: Zeal Optics, Inc., Boulder, CO (US)

(72) Inventors: John C. Sanchez, Boulder, CO (US); Carl L. Walker, Boulder, CO (US)

(21) Appl. No.: 14/602,139

(22) Filed: Jan. 21, 2015

Related U.S. Application Data
(60) Provisional application No. 61/930,277, filed on Jan. 22, 2014.

(51) Int. Cl. A61F 9/02

(52) U.S. Cl. CPC A61F 9/029; A61F 9/026

(57) ABSTRACT

An all-weather goggle includes a lens with an arcuate shape. The all-weather goggle includes a frame that includes an inner frame with a first elasticity and an outer frame coupled to the inner frame, the outer frame comprising a second elasticity that is less than the first elasticity. The inner frame is positioned proximate a user's face. The outer frame is coupled to the inner frame and is configured to removably receive the lens. The outer frame maintains the arcuate shape of the lens when the lens is worn by the user and when the lens is not worn by the user. A first portion of the all-weather goggle is made of thermoplastic polyurethane and a second portion of the all-weather goggle is made of a thermoplastic polyamide.

### PRIORITY CLAIM

The present application claims priority to and the benefit of U.S. Provisional Patent Application Ser. No. 61/930,277 filed Jan. 22, 2014 and titled All-Weather Goggle, the disclosure of which is incorporated in its entirety by this reference.

### FIELD

The present application relates to all-weather goggles for use in sports, outdoor activities, military, and safety related applications.

### BACKGROUND

The present application relates to sports goggles, more particularly an all-weather goggle having a front frame of one material and a rear frame of another material.

All-weather goggles are more generally described in U.S. Pat. No. 8,356,895 that issued on Jan. 22, 2013 and assigned to the assignee of this invention, the disclosure of which is incorporated herein in its entirety by this reference for all purposes.

Goggles have been used to protect a user's eyes while participating in various activities, including outdoor activities and sports, such as skiing and snowboarding, snowmobiling, snowshoeing, shooting, motorcycle and all-terrain vehicle riding, and similar events, as well as military activities and safety goggles when a user requires eye protection.

When used for outdoor activities, goggles protect the user's eyes from inclement weather conditions, such as cold weather and wind. In addition, the goggles protect against snow, ice, rain, and blowing particulate matter, such as sand. Further, goggles, if so configured, may provide protection from sunlight, ultraviolet radiation, and provide improved contrast and reduced glare from the light reflected off various objects.

While most goggles provide either a greater or lesser degree of protection from physical elements, such as the weather and blowing particulate matter, some previous goggles are designed to work best in a particular light condition. If light conditions change, such as with a change in cloudiness or daylight as the day passes, a particular goggle that is well-suited for one light condition becomes less effective in another light condition as the ambient light changes throughout a day.

Instead of relying on only one goggle with a limited range of ability, the user might choose to bring several goggles or replacement lenses, each goggle or lens designed to work best in a different type of light condition. While this solves the problem of having a goggle and/or lens available that works best in a given light condition, it requires a user to dedicate space and weight to carrying one or more extra goggles and/or lenses. In addition, such a solution requires a user to stop and change the goggles or lenses he or she uses, which may not be practical in certain locations and weather conditions.

Further, goggles worn while skiing, snowboarding, snowmobiling, etc. protect the user from a variety of dangers inherent to alpine environments from adverse weather conditions to physical harm in a crash or fall. However, these goggles must remain flexible to remain comfortable over long periods of time and conform to different head and face sizes.

This compromise of strength and rigidity for flexibility presents a problem and safety hazard for skiers or snowboarders. Further, flexible frame structures compromise the designed base curve of the goggle lens when the frame is deformed from its original shape, essentially bending the lens and distorting optical performance. For example when the goggle strap is over tightened by the user, the goggle frame is stretched around the user's head and the contained lens which is built to provide clear clean optics in a specific curve is bent into another curve.

Therefore, a need exists for a goggle that provides a user with enhanced visual acuity and contrast in a variety of light conditions. Such a goggle should be light-weight, flexible, scratch and impact resistant.

In addition, a need exists for a goggle that provides protection from impact during use and is sufficiently rigid to maintain an arcuate shape and/or base curve of a lens when it is worn while also retaining sufficient flexibility to comfortably conform to a user's face.

### SUMMARY

Embodiments of the lens disclosed herein are adapted for use with a frame that is configured to receive the lens and position the lens proximate a user's face and eyes. The frame includes various types of securing devices to hold the frame about the user's head, such as, for example, arms, as in various safety glasses, straps like those used in ski goggles, and other methods of affixing goggles to the user's head. Other embodiments include a lens affixed in the form of a visor to headwear such as helmets used by military pilots and other occupations in which a lens that adapts to ambient or changing light conditions is desirable.

Another embodiment of the all-weather goggle comprises frame that includes a first portion made of thermoplastic polyurethane and a second portion made of a thermoplastic polyamide.

Another embodiment of the all-weather goggle comprises a frame that includes an inner frame with a first elasticity and an outer frame coupled to the inner frame, the outer frame comprising a second elasticity that is less than the first elasticity.

Yet another embodiment of the all-weather goggle includes a lens with an arcuate shape. An inner frame is positioned proximate a user's face. An outer frame is coupled to the inner frame and is configured to removably receive the lens. The outer frame maintains the arcuate shape of the lens when the lens is worn by the user and when the lens is not worn by the user.

Methods of manufacturing various embodiments of the lens are disclosed herein, as well as various frames and other methods of positioning the lens proximate to a user's eyes are disclosed.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C” and “A, B, and/or C” means A alone, B alone, C alone, A and B together, A and C together, B and C together, or A, B and C together.

Various embodiments of the present inventions are set forth in the attached figures and in the Detailed Description as provided herein and as embodied by the claims. It should be understood, however, that this Summary does not contain all of the aspects and embodiments of the one or more present inventions, is not meant to be limiting or restrictive in any manner, and that the invention(s) as disclosed herein is/are and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

To further clarify the above and other advantages and features of the one or more present inventions, reference to specific embodiments thereof are illustrated in the appended drawings. The drawings depict only typical embodiments and are therefore not to be considered limiting. One or more embodiments will be described and explained with additional specificity and detail through the use of the accompanying drawings in which:

FIG. 1 a cross-section A-A of the lens in FIG. 2;

FIG. 2 is front-view of an embodiment of a lens disclosed herein;

FIG. 3 is a front iso-view of the lens in FIG. 2;

FIG. 4 is a rear iso-view of the lens in FIG. 2;

FIG. 5 is a rear-view of the lens in FIG. 2;

FIG. 6 is a top-view of the lens in FIG. 2;

FIG. 7 is a bottom-view of the lens in FIG. 2;

FIG. 8 is a front-view of an embodiment of a frame configured to receive the lens in FIG. 2;

FIG. 9 is a side-view of the frame and lens in FIG. 8;

FIG. 10 is a top/rear-view of the frame and lens in FIG. 8;

FIG. 11 is a bottom/rear-view of the frame and lens in FIG. 8.

FIG. 12 is a front perspective view of an embodiment of a goggle that includes an inner frame and an outer frame.

FIG. 13 is another front perspective of the goggle in FIG. 12.

FIG. 14 is a rear perspective view of the goggle in FIG. 12.

FIG. 15 is a rear view of the outer frame of the goggle in FIG. 12.

FIG. 16 is a top perspective view of the inner frame of the goggle in FIG. 12.

FIG. 17 is a top perspective view of a right front cover of the goggle in FIG. 12.

FIG. 18 is a top perspective view of a left front cover of the goggle in FIG. 12.

FIG. 19 is a top perspective view of a top cover of the goggle in FIG. 12.

FIG. 20 is a front perspective view of a battery cover and battery of the goggle in FIG. 12.

FIG. 21 is a top perspective view of an electronics cover of the goggle in FIG. 12.

FIG. 22 is a front perspective view of the inner frame of the goggle in FIG. 12.

FIG. 23 is a top perspective view of the inner frame of FIG. 22.

FIG. 24 is an exploded view of the camera and associated electronic components of the goggle in FIG. 12.

FIG. 25 is an exploded view of the camera and associated electronics of the goggle of FIG. 12.

The drawings are not necessarily to scale.

### DETAILED DESCRIPTION

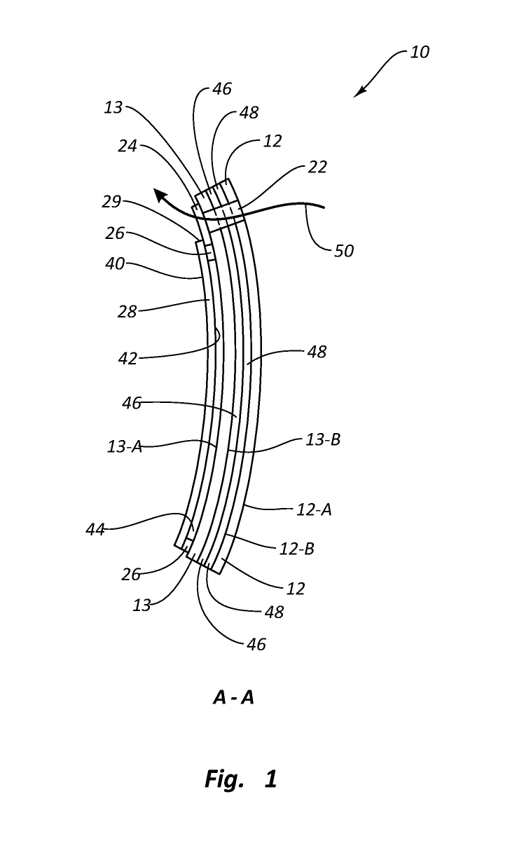

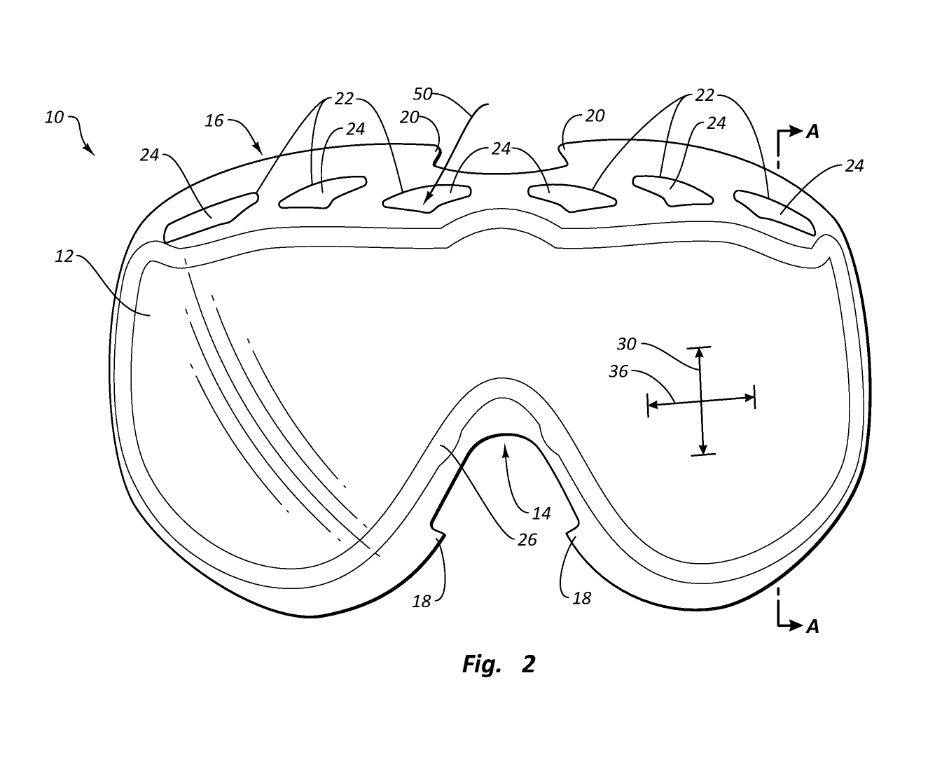

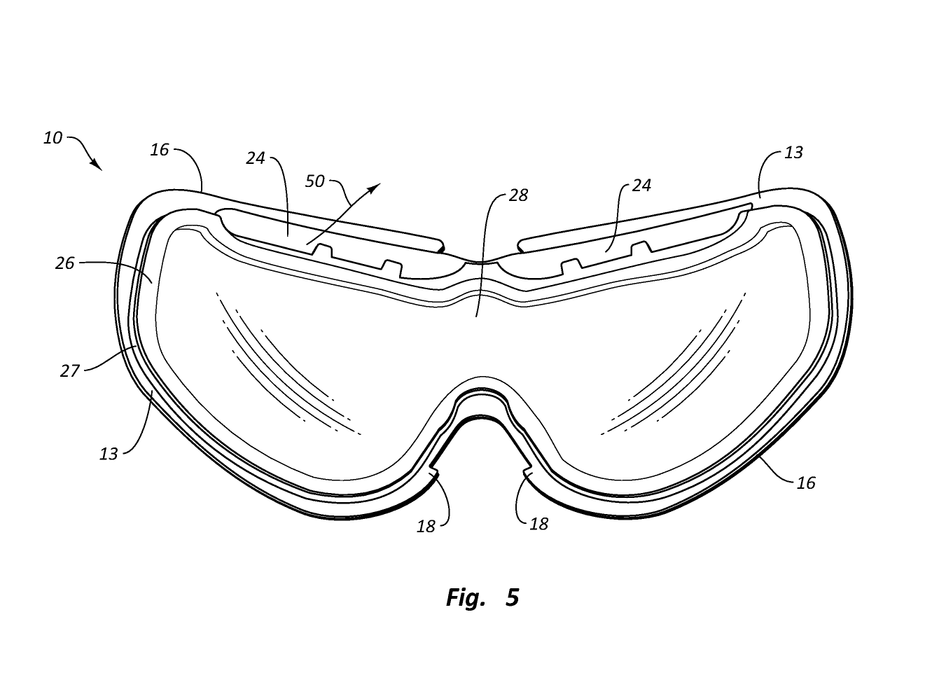

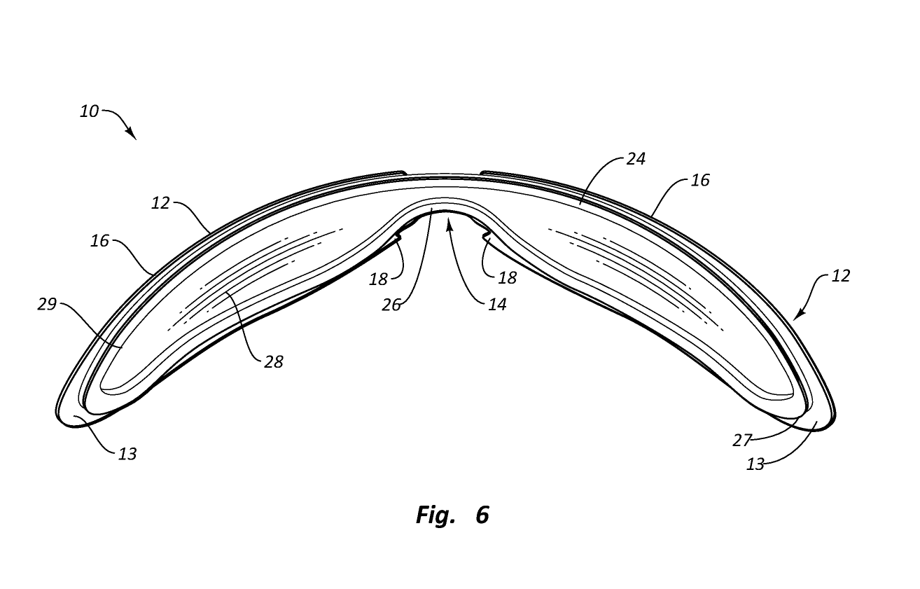

Illustrated in FIGS. 2 through 7 are several views of an embodiment of a lens disclosed herein. In addition, FIG. 1 illustrates a cross-section A-A (not to scale) of the lens illustrated in FIGS. 2 through 7.

Turning first to FIG. 1, an embodiment of a lens 10 includes a first layer 12 made of a transparent thermoplastic material. One embodiment of the first layer includes a clear material, i.e., without tint; another embodiment includes a selected color of tint, which includes gray and/or other colors of the spectrum. The first layer 12 is the outer layer of the lens 10 that is exposed to the ambient environment. Optionally, the first layer 12 includes a scratch resistant coating (not shown) applied to the outer first surface 12-A. The first layer 12 also includes an inner first surface 12-B opposite the outer first surface 12-A.

The lens 10 also includes a second layer 13 made of a transparent thermoplastic material. An embodiment of the second lens includes a clear material, i.e., without tint; another embodiment includes a selected color of tint, which includes gray or other colors of the spectrum. The second layer 13 is the inner layer of the lens 10 that is proximate a user's eyes when the lens 10 is received by a frame that is positioned about the user's head. Like the first layer 12, the second layer 13 includes an outer second surface 13-A, but this outer second surface is positioned proximate the user's eyes instead of the ambient environment. The second layer 13 also includes an inner second surface 13-B.

The first layer 12 and the second layer 13 are typically made from a flat polycarbonate sheet. Other transparent thermoplastic materials that provide the desired characteristics as described below will also work. As will be discussed below, the sheet is thermoformed to have a curvature along both a long axis and a short axis to provide a spherical lens, thereby reducing distortion of the light that is transmitted through the lens and, to a degree, increasing clarity and visual acuity of objects that a user views through the lens.

A first film or layer 48 is affixed to first layer 12. While the first film 48 is shown affixed to the inner first surface 12-B of the first layer 12, it is optionally affixed to the inner second surface 13-B of the second layer 13. The first film 48 is a transparent, polarized film, i.e., a film that is selected and configured to filter and permit those wavelengths of light oriented in the same direction as the filter in the polarized film to be transmitted through the first film 48. As a non-limiting example, if the polarizing filter in the first film 48 is oriented towards the horizontal, i.e., parallel to a user's eyes, only those wavelengths of light oriented in the same direction (horizontal) will pass through the first film; wavelengths of light oriented in any direction other than horizontal (such as vertical or at a diagonal to the horizontal filter) will be blocked by the first film 48. It will be understood, of course, that the orientation of the filter direction of the first film 48 can be selected to a desired orientation. An advantage that polarized films, such as first film 48, provide is that they significantly reduce glare from the light reflected off various objects. This is particularly significant for those objects that typically are highly reflective, including, but not limited to, snow, water, glass, chrome, metals, and other similar color and types of objects. When a user views an object through a polarized film, the polarized film reduces the glare caused by reflected light off of that object, thereby allowing the user to more easily view the object and other objects near the reflective object. In reducing glare, eye strain that a user endures may be reduced for the user as well as other associated ailments associated with eye strain, such as headaches. Furthermore, reducing glare diminishes the likelihood and/or the severity of temporary or permanent eye damage caused from burns and similar ailments that lead to snow-blindness.

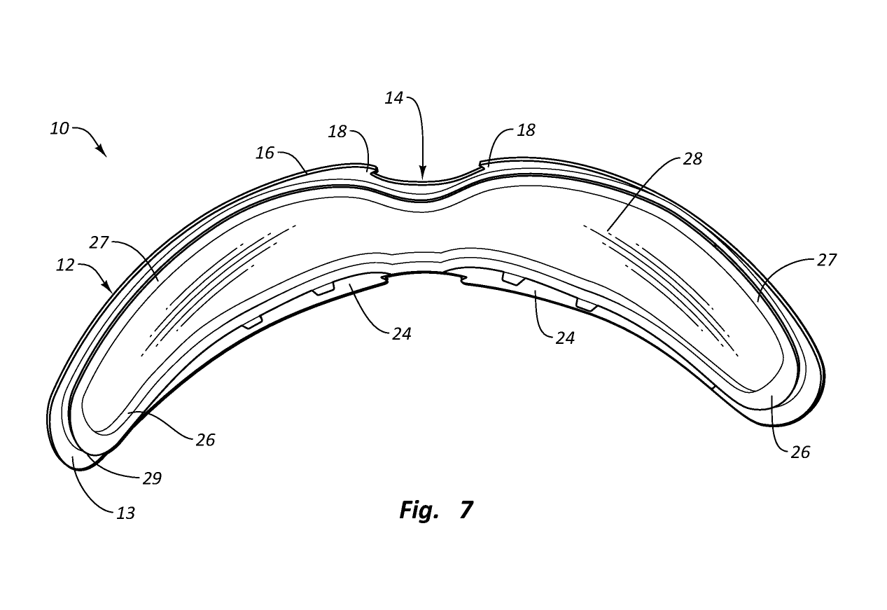

The lens 10 also includes a transparent second film, a photochromatic film 46. In FIG. 7, the photochromatic film 46 is affixed to the inner second surface 13-B of the second layer 13 and the first film 48, but it is understood that the photochromatic film 46 can alternatively be affixed to the inner first surface 12-B of the first layer 12 with the first film 48 affixed to the inner second surface 13-B of the second layer 13. The photochromatic layer 46 automatically adjusts to vary the amount of light that is transmitted through the photochromatic film 46 in response to the amount of ultraviolet radiation received by the photochromatic film 46. As one, non-limiting example, as the amount of ultraviolet radiation received by the photochromatic film 46 increases, the photochromatic film 46 becomes darker and thereby decreases the amount of visible light, which is typically proportional to the amount of ultraviolet radiation received by the photochromatic film 46, that is transmitted through the photochromatic film 46. Conversely, when the amount of ultraviolet radiation received by the photochromatic film 46 decreases, the photochromatic film 46 becomes relatively less dark, or lighter, thereby increasing the amount of visible light that is transmitted through the photochromatic film 46. In each instance, the photochromatic film adjusts automatically to the prevailing visible light conditions, thereby enhancing what a user sees through the lens 10.

The photochromatic film 46 optionally includes various tints or coloring agents that block selected wavelengths of light. As known, visible (white) light comprises a spectrum of colors at different wavelengths. Tinted or colored films selectively block certain wavelengths of light from being transmitted through the photochromatic film 46. In doing so, the remaining light transmitted through the photochromatic film 46 enhances the contrast between objects. An example of selecting such a tint is the use of rose-gray or purple-gray, or a purple-rose-gray tint, that is selected to enhance the contrast between lighter and darker objects in low-light or flat-light conditions during cloudy or partly-cloudy days. Of course, tints and coloring agents of other colors within the spectrum of available colors can be selected, including, but not limited to, red; red-orange; persimmon; yellow; blue; green; and others. For example, such tints enhance the contrast between uneven surfaces on snow while skiing, thereby allowing a user to see the uneven surface with sufficient time to react and, therefore, increasing the safety and the enjoyment of the user.

Another example of a tint suitable for use with the photochromatic film 46 includes one that changes color in response to the received ultraviolet light, just as the photochromatic film 46 increases or decreases in darkness (or grayness) when exposed to ultraviolet light. As a non-limiting example, the tint of the photochromatic film 46 may change from a yellow-gray to a rose-gray, rose-purple-gray, and/or purple-gray, when exposed to increasing amounts of ultraviolet radiation. Of course, different combinations of tints that adjust to ultraviolet can be used, including those within the spectrum of available colors, such as red; red-orange; persimmon; yellow; blue; green; and others. By using a tint that adjusts in color to ultraviolet light, the range of visible light conditions in which the lens with the photochromatic film 46 increases, thereby making the lens more adaptable to a variety of light conditions.

As an example, a lens 10 that includes the polarized film 48 and the photochromatic film 46 tinted with a rose-purple-gray color adjusts to the ambient light conditions in response to the amount of ultraviolet light received at that photochromatic film 46 and thereby allows varying amounts of visible light to be transmitted through the lens 10. For instance, in very bright sunlight, such as a cloudless day, the photochromatic film 46 would be very dark in response to the large amount of ultraviolet radiation received at the photochromatic film 46. Combined, the polarized film 48 and photochromatic film 46 allow only from about 6% to about 20% and, more preferably, about 10% to about 16%, of the available ambient light to pass through the lens 10 in bright ambient light conditions.

Conversely, on a very cloudy day or late in the afternoon or early evening while the sun is setting, the ambient light conditions would be very low and, consequently, the amount of ultraviolet radiation received at the photochromatic film 46 would be very low. As a result, the photochromatic film 46 would be relatively less dark and, consequently, allow a relatively larger amount of ambient light to pass through the photochromatic film 46. In this example, the polarized film 48 and the photochromatic film 46 combine to permit about 26% to about 40% and, more preferably, about 30% to about 36% of available ambient light to pass through the lens 10 in relatively low ambient light conditions.

Thus, as this example illustrates, an embodiment of the lens 10 allows about 6% to about 40% and, more preferably, about 10% to about 36% of the available ambient light to pass through the lens depending on the ambient light conditions and the ultraviolet light that reaches the photochromatic film layer 46. As it will be understood, any amount of ambient light between the top and bottom of these ranges will be transmitted through the lens 10 depending on the amount of ultraviolet radiation received at the photochromatic film 46.

Another example of the lens 10 includes the polarized film 48 and the photochromatic film 46 having a tint that changes color in addition to adjusting its relative darkness in response to the amount of ultraviolet radiation that reaches the photochromatic film 46. For example, in the very bright sunlight conditions described above, the tint optionally is a rose-purple-gray that allows, in combination with the polarized film 48, only allow from about 6% to about 20% and, more preferably, about 10% to about 16%, of the available ambient light to pass through the lens 10 in bright ambient light conditions.

In contrast, in low-light conditions such as a cloudy day or a late afternoon or evening, the photochromatic film 46 would receive relatively little ultraviolent radiation. Not only would the photochromatic film 46 become relatively less dark and allow more light to pass through the lens 10, the tint would also change color, from rose-purple-gray in bright ambient light conditions to a yellow-gray in low ambient light conditions. In other words, the different color tint would block a different wavelength of light. Because the wavelength of available light changes with the amount of ambient light—most notably, shorter wavelength blue light become more prevalent in the late afternoon and early evening, a tint that adjusts color to the ambient light conditions would increase the contrast between objects as viewed through the lens 10 by a user. In this instance, the yellow-gray tint of the photochromatic lens 46 would allow from about 33% to about 47% and, more preferably, from about 37% to about 43% of available ambient light to pass through the lens 10 in relatively low ambient light conditions.

Thus, as this example illustrates, this embodiment of the lens 10 allows about 6% to about 47% and, more preferably, about 10% to about 43% of the available ambient light to pass through the lens depending on the ambient light conditions and the amount of ultraviolet radiation that reaches the photochromatic film layer 46. As it will be understood, any amount of ambient light between the top and bottom of these ranges will be transmitted through the lens 10 depending on the amount of ultraviolet radiation received at the photochromatic film 46. This embodiment therefore provides an optimum range of light to be transmitted through the lens 10 over a wider range of light conditions than the previous example. In addition, it will be understood that the adaptable tint can be selected for a variety of colors and light conditions, not just those recited in these examples. Of course, different combinations of tints that adjust to ultraviolet radiation can be used, including those within the spectrum of available colors, such as red; red-orange; persimmon; yellow; blue; green; and others.

Another embodiment of the lens includes a photochromatic dye applied to a surface of at least one of the first layer and the second layer to form an equivalent of a film on the surface of the first layer and/or the second layer. Alternatively, the photochromatic dye can be incorporated into the material of at least one of the first layer and the second layer. The photochromatic dye optionally includes a tint of the desired type as described above.

A lens of the types described above is manufactured as follows. The first layer 12 and the second layer 13 are made from a transparent thermoplastic resin, such as polycarbonate, that is extruded in a flat sheet. The polarized film 48 has a bonding agent or chemical fixant applied to one or both sides of the polarized film 48. Conversely, the bonding agent or chemical fixant (not shown) can be applied to one or both of the inner first surface 12-B of the first layer 12 and the inner second surface 13-B of the second layer 13. Of course, the bonding agent can also be applied to both the polarized film 48 and the first layer 12 and the second layer 13. The bonding agent can by any type of transparent bonding agent known in the art that can be used to adhere the polarized film 48 to the first layer 12, including, but not limited to, UV cured, heat cured, pressure cured, and typical time/air cured bonding agents. The result is that the polarized film 48 forms a laminate with the first layer 12.

The photochromatic film 46 is applied in the same manner as the polarized film 48 to the first layer 12, the polarized film 48, and the second layer 13. In other words, the same bonding agent as that used with the polarized film 48 is applied to one or more of the photochromatic film 46, polarized film 48, first layer 12, and second layer 13 so that the photochromatic film 46 forms a laminate with the polarized film 48 and the first layer 12.

The second layer 13 is then affixed to the laminate of the photochromatic film 48, polarized film 46, and first layer 12 to form a flat lens 10. It will be understood that the order in which the various films and layers are joined to form the laminated lens 10 can be varied as desired. Further, the bonding agent of all the layers can be activated at the same time once each of the polarized film 46, photochromatic film 48, first layer 12, and second layer 13 are laminated to each other, or the bonding agent can be activated in sequence as each of the aforementioned is applied. Finally, the bonding agent can be applied to the entire surface of each of the aforementioned elements or it may be applied to selected portions only of the polarized film 48, the photochromatic film 46, and the first and second layers 12, 13, respectively.

In applying the polarized film 48 and the photochromatic film 46, care must be taken to avoid introducing lines, wrinkles, air bubbles, and other defects into the laminated lens 10. The polarized film 48 and photochromatic film 46 should provide a smooth surface substantially free of such defects within the tolerance level associated with the manufacturing process.

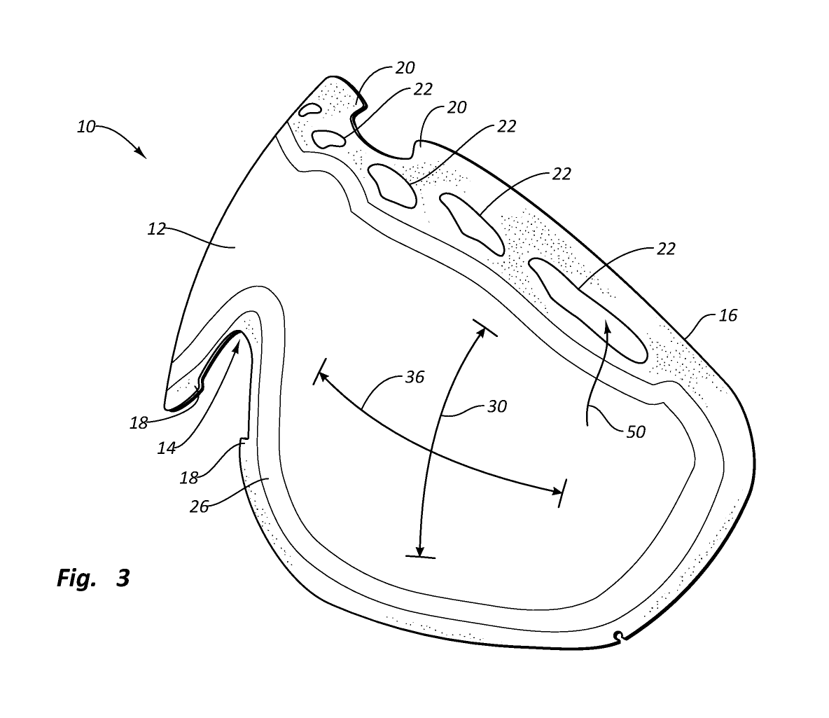

The flat laminated lens 10 is then placed into a mold of a selected shape and thermoformed at a selected pressure and temperature to provide the desired shape and curvature. For example, an embodiment of the lens 10 can be formed as a large, single lens with dimensions of from about 185 millimeters to about 235 millimeters in width and, more preferably, from about 195 millimeters to about 225 millimeters, corresponding to the horizontal axis 36 illustrated in FIGS. 2 and 3. The embodiment of the lens 10 has a height of from about 80 millimeters to about 130 millimeters and, more preferably, from about 90 millimeters to about 120 millimeters, corresponding to the vertical axis 30 illustrated in FIGS. 2 and 3. The lens 10 has a thickness of from about 0.4 millimeters to about 2.0 millimeters and, more preferably, from about 0.6 millimeters to about 1.8 millimeters.

The flat laminated lens 10 is selectively provided with an arcuate shape or curvature in one or both of the vertical axis 30 and the horizontal axis 36 illustrated in FIGS. 2 and 3. The minor, or vertical axis 30, extends from the forehead downward toward the user's cheeks, while the major, horizontal axis 36, extends generally from one side to the other side of the user's head in alignment with the user's eyes. A lens 10 that includes an arcuate shape or curve is desirable because the curve, especially a curve that is similar to the natural curve of the human eye, can increase visual acuity and clarity by minimizing the distortion of light that may occur as it passes through a flat lens.

Typically, a curve is described as having either a radius of curvature or a given base curve number, which has a numerical relation to the radius of curvature. Specifically, the base curve number is calculated by dividing the radius of curvature in millimeters into 530.

The radius of curvature in either the horizontal axis 36 and the vertical axis 30 extends in a range of from about 44 millimeters to about 265 millimeters, which qualitatively corresponds to a significant curve to a nearly flat, or insignificant curve, and corresponds to a base curve of from about 12 to about 2. More preferably, the curve in the horizontal axis 36 ranges from about a 3 base curve to about a 9 base curve and, more preferable still, from about a 5 base curve to about a 7 base curve. The curve in the vertical axis preferably ranges from about a 1 base curve to about a 7 base curve and, more preferable still, from about a 3 base curve to about a 5 base curve. Of course, it will be understood that the curve in either of both of the horizontal axis 36 and the vertical axis 30 need not be constant across the entire width or height of the lens 10 and, in fact, can vary across the width and the height of the lens 10 as desired.

As mentioned, the flat lens 10 comprised of the first layer 12, the polarized film 48, the photochromatic film 46, and the second layer 13 is thermoformed as a single piece in a mold of a selected shape at a selected pressure and temperature to create an arcuate shape of a selected curvature. Because the mold provides a curved or arcuate shape, the process is carefully controlled to minimize or eliminate any visible stretching, wrinkling, cracking or crackling, lines, or other distortions in either or both the polarized film 48 and the photochromatic film 46 as pressure is applied and the polarized film 48 and photochromatic film 46 with the first layer 12 and the second layer 13 each increase in length as the lens 10 assumes a curved shape under the heat and pressure of the mold. Such a thermoforming process allows a high quality, relatively thinner and more flexible lens 10 to be produced at less cost and complexity than the typical injection molding process normally used to create a curved lens.

The result is a thin lens 10, thinner than a lens typically formed through injection molding, that has less weight and greater flexibility. The flexibility of the lens 10 allows a user to easily remove and replace the lens 10 in a frame, as will be described below. In addition, the flexible lens 10 is resistant to breaking under impacts and other sudden forces unlike more rigid and thicker lenses. Further, the light weight of the lens relative to other lenses creates a more comfortable wearing experience for the user, with less weight, pinching, and sliding of the frame and lens around the user's nose and head, an important factor when worn for extended periods or in cold and/or inclement weather. It should be noted that the lens 10 retains its flexibility in a wide range of temperature conditions, including all those that it might normally be exposed to, such as from about −30 degrees Fahrenheit to about 120 degrees Fahrenheit.

Another embodiment of the lens includes those that are formed by having at least one of the first layer and the second layer formed by injection molding and/or casting to the desired shape and/or curvature rather than thermoforming a flat first layer and flat second layer. The polarized film and/or the photochromatic film can be applied as a laminate as described above to at least one of the injection molded or cast first layer and the second layer.

Examples of the above described lens are manufactured according to instructions from Zeal Optics, Inc., 4843 Pearl Street 1A, Boulder, Colo., 80301 by Mitsubishi Chemical Corporation, 14-1 Shiba 4-chome, Minato-ku, 108-0014, Tokyo, Japan.

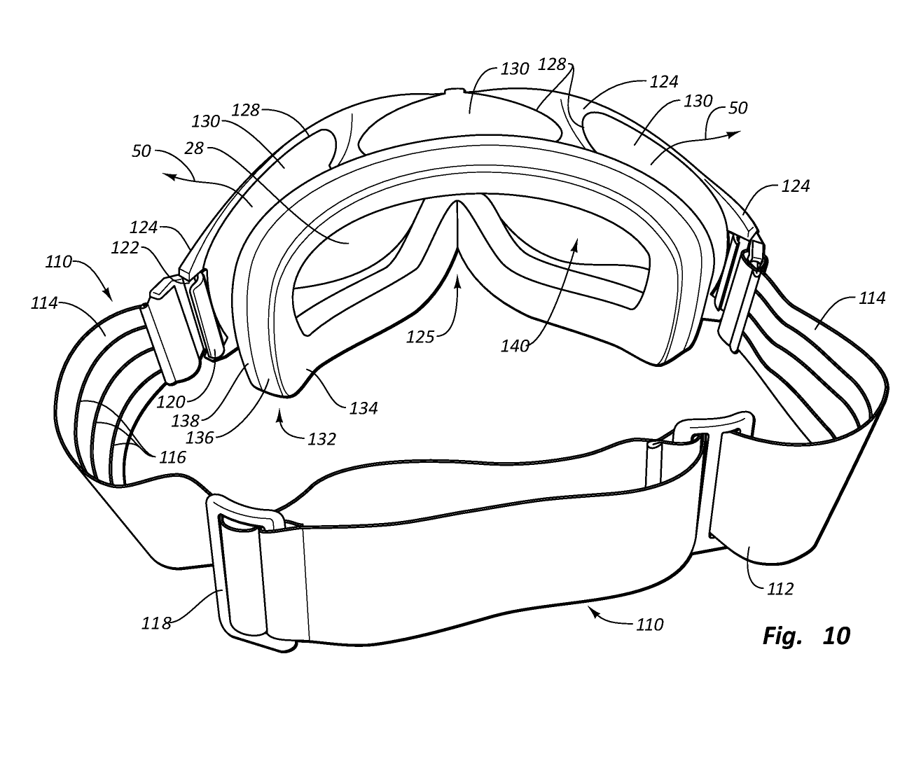

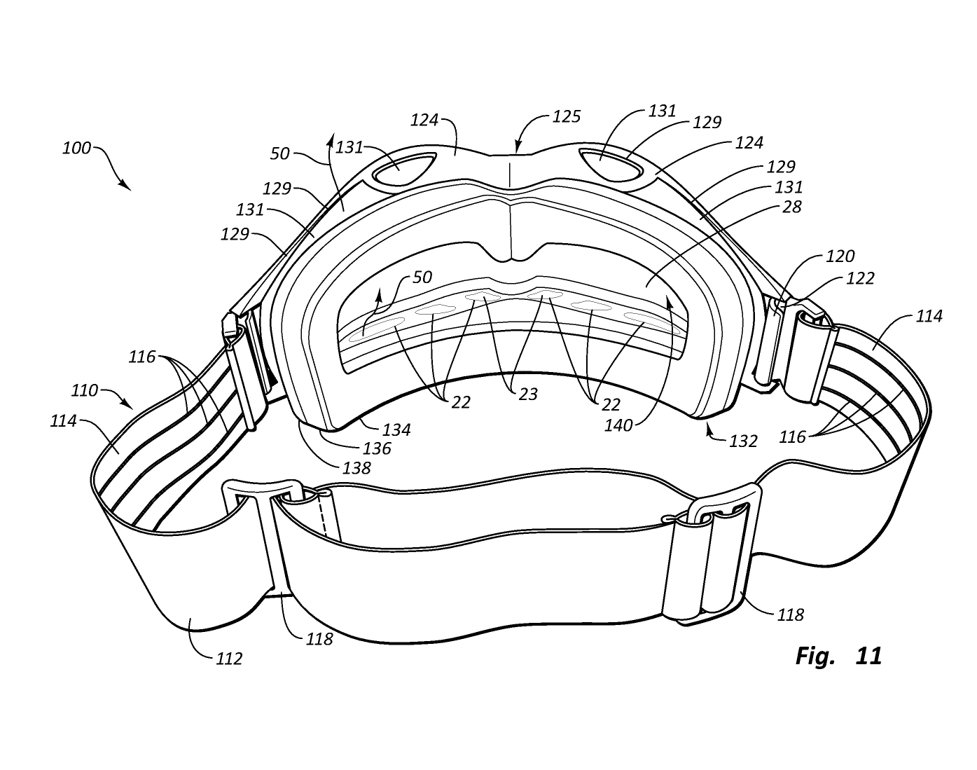

Optional elements of the lens are further illustrated in FIG. 1. For example, the lens 10 can include one or more vents 22, also illustrated in FIGS. 2 through 7, that allow venting air illustrated as flow arrow 50 to pass through the lens 10 from an exterior of the lens 10 into an interior portion 140 of the frame 124 (illustrated in FIGS. 10 and 11). A porous foam 24 can be affixed with a bonding agent as known in the art to the outer surface 13-B of the second layer 13 and proximate to the vent 24. The porous foam 24 allows venting air 50 to pass through the vent(s) 22, while at the same time preventing or limiting the entrance of dust, snow, dirt, other particulate matter, and the like from entering into the interior space 140 of the frame 124 through the vent(s) 22. The venting air 50 passes through the porous foam 24, thereby being slowed and dispersed by the foam in the interior space 140. The venting air 50 acts to prevent and/or minimize any condensation that might otherwise buildup in the interior space 140 of the frame 124.

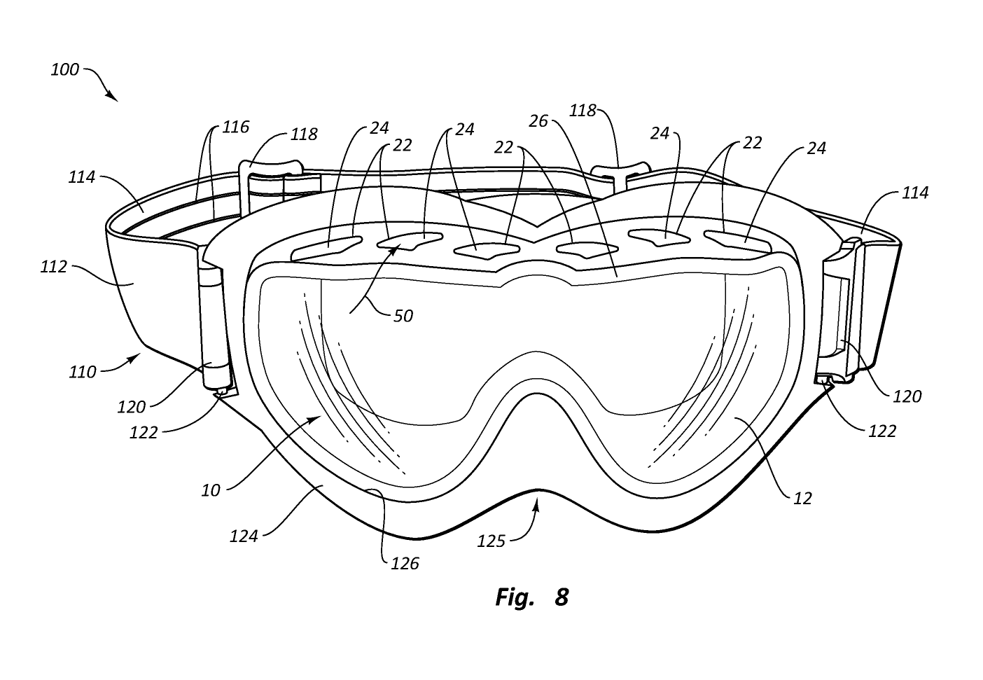

The venting air 50 passes through the interior space 140, at least partially replacing the air therein and aiding the evaporation of any condensation in the interior space 140, such as condensation that might otherwise form on the interior of the lens 10. A portion of the venting air 50 then passes through a porous upper frame vent foam 130 and out the upper frame vent 128 to the exterior of the frame 124. Similarly, a portion of the venting air 50 passes through a porous lower frame vent foam 131 and out the lower frame vent 129 to the exterior of the frame 124. This process is best illustrated in FIGS. 8, 10, and 11. The upper frame vent foam 130 and lower frame vent foam 131 allow venting air 50 to pass through the upper frame vent 128 and lower frame vent 129, while at the same time preventing or limiting the entrance of dust, snow, dirt, other particulate matter, and the like from entering into the interior space 140 of the frame 124 through the upper frame vent 128 and the lower frame vent 129.

The lens 10 also optionally includes an anti-fog lens 28 spaced apart from the lens 10, in particular spaced apart from the second outer surface 13-A of the second layer 13, to form an air space 44 between the anti-fog lens 28 and the lens 10. Thermal gasket seals 26, such as those made from elastomers, polymers, and other similar, non-porous materials, are bonded to the anti-fog lens 28 and the second outer surface 13-A of the second layer 13 with a bonding agent, double sided tape, and other similar methods known in the art. The thermal gasket seals 26 limits or prevents the entrance of external air into the air space 44.

The air space 44 creates a thermal barrier between the lens 10, which often is exposed to inclement weather such as below freezing temperatures at the first outer surface 12-A of the first layer 12. In so doing, the air space 44 decreases the potential for condensation, such as sweat and water vapor present in the interior space 140 near a user's face from cooling and condensing on the anti-fog lens 28 or the second outer surface 13-A of the second layer 13. An anti-fog coating can optionally be applied to the anti-fog lens 28 to improve the anti-fogging characteristics of the anti-fog lens 28.

The anti-fog lens 28 is typically formed of a flat, transparent thermoplastic, such as one of acetate, polycellulose acetate, cellulose acetate, cellulose acetobutyrate, and a cellulose acetopropionate. The anti-fog lens 28 should have the characteristic of being light in weight and flexible so that when it is affixed via the thermal gasket seals 28 to the lens 10, the lens 10 retains its overall flexibility and relatively light weight to allow comfortable wear and its ability to be replaceable.

Other optional features of the lens 10 are better illustrated in FIGS. 2 through 7. For example, the embodiment of the lens 10 illustrated in FIGS. 2 through 7 includes a lens edge 16 configured to register with a groove 126 in the frame 124 so that the lens 10 is retained in the frame 124. The lens 10 includes nose tabs 18 and forehead tabs 20 along the lens edge 16 that are configured to register with structures in the frame 124 to increase the ability of the frame 124 to retain the lens 10 throughout a variety of conditions and environments, including potentially sudden and/or violent movements and impacts. The lens 10 also includes a nose portion 14 configured to register with the nose portion 125 of the frame 124 as well as a user's nose.

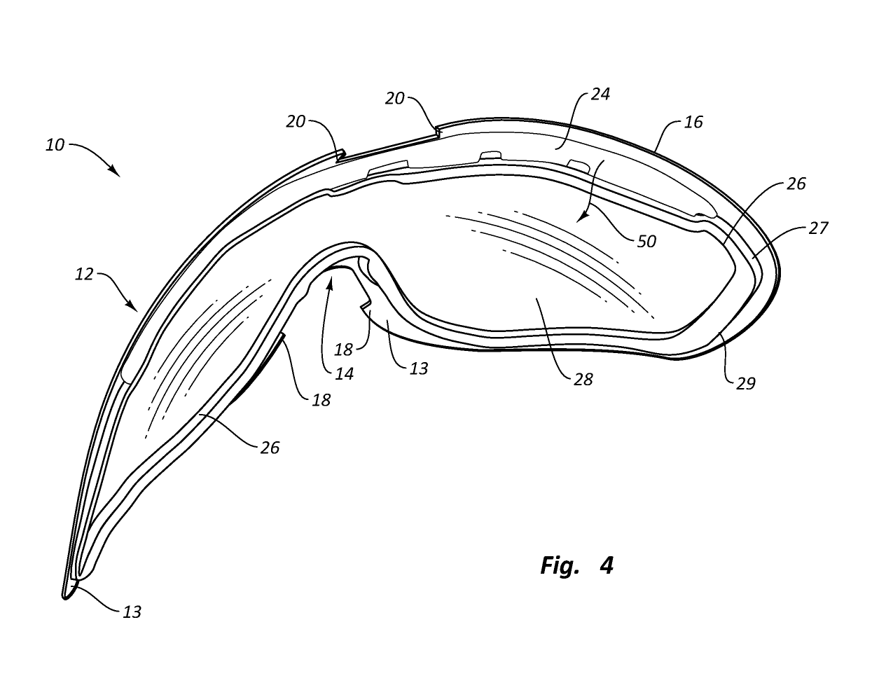

As best seen in FIG. 4, the anti-fog lens 28 has an anti-fog lens edge 29 that registers with the thermal gasket seal edge 27.

In the embodiment of the lens 10 illustrated, the lens 10 includes a plurality of vents 22 across an upper portion of the lens 10. The vents 22 can be of a variety of shapes and sizes and therefore are not limited to the shapes and sizes illustrated. A single, continuous strip of porous foam 24 is affixed to the second outer surface 13-A of the second layer 13 that covers each of the plurality of vents 22. While a single, continuous strip of porous foam 24 is illustrated, a plurality of separate porous foam pieces or strips 24 to cover the vents 22 falls within the scope of the disclosure.

An embodiment of a goggle 100 that includes a frame 124 configured to retain and position the lens 10 about a user's head and proximate the user's eyes is illustrated in FIGS. 8 through 11. The frame 124 is made from a flexible thermoplastic, such as molded polyurethane, that allows the frame 124 to flex around a user's face to better create a seal against the user's face to prevent ambient environmental factors, such as dust, wind, cold, and snow from easily entering the interior space 140. The frame portion optionally includes a nose portion 125 that allows the frame 124 to rest upon the bridge of the user's nose.

The terms goggle, goggles, and pair of goggles are used here synonymously to refer to a structure that has a frame sized to hold and configure to position a transparent lens, with or without tint, in front of the user's eyes to offer protection of some type to the user's eyes. While some goggle, goggles, and/or pair of goggles may have two separate lenses, one for positioning in front of each eye of the user, the embodiments herein illustrated present a goggle, goggles, and/or pair of goggles that have a single lens structure that extends between and in front of both eyes of the user.

As illustrated, the lens 10 is retained within the frame 124, the lens edge 16 configured to register in a groove 126 in the frame 124 to securely retain the lens 10 within the frame 124 in most situations other than when a user wishes to remove the lens 10, such as when the user wishes to replace the lens 10. As discussed previously, the lens edge 10 can include nose tabs 18 and forehead tabs 20 configured to register with reciprocal features in the groove 126 of the frame 124 to increase the ability of the frame 124 to secure the lens 10 from undesired removal.

The vents 22 allow venting air 50 to flow through porous foam 24 into the interior space 124 defined by the lens 10, the frame 124 and the user's face as discussed above.

The frame 124 includes a securing device 110 for securing the lens 10 proximate the user's face and eyes and, more generally, about the user's head. The securing device 110 illustrated in FIGS. 8 through 11 is a woven, super Jacquard strap which stretches preferentially in one direction—in this instance along the long axis of the strap, i.e., horizontally, but exhibits little to no stretch in the vertical direction. For example, an exemplary embodiment of the super Jacquard weave strap is one that stretches from about 60% to 90% beyond its original length in the horizontal direction, but only stretches from about 10% to about 30% of its original height in the vertical direction, while returning approximately to its original length and width when the stretching force is removed. Of course, straps made of other materials, including strings, rubber, elastic, other weaves of material, and the like fall within the scope of the disclosure. Additionally, in other embodiments, such as safety or sunglasses, the securing device include arms (adjustable, flexible, and rigid), hinges (such as those that allow a lens to flip down from a safety helmet or other headwear), attachments for visors, such as in a military pilot's helmet, and other securing devices known in the art. The securing device 110 in this instance have a clip 120 configured to register with a post 122 on the frame 124, but other types of securing devices secured to the frame 124 exist, as discussed above. The clip 120-post 122 arrangement allows the securing device 110 in this embodiment to adjust to whether a user is wearing the goggle 100 with or without a helmet.

The securing device (strap) 110 illustrated includes a strap outer side 112 and a strap inner side 114 opposite the strap outer side 112. The strap inner side 114 optionally includes gripping material 116 made of rubber, elastomer, or other thermoplastic positioned on the strap inner side to provide increased friction with the strap inner side 114 against a helmet or a user's head and, thereby, reducing the likelihood of the securing device 110 from slipping or otherwise moving out of position. While the gripping material 116 is illustrated as three lines approximately equidistant apart across the height of the securing device 110, other configurations of gripping material, such as patterns of dots and other geometric patterns, fall within the scope of the disclosure.

Finally the securing device 110 optionally includes a means of adjusting the length of the securing device 110 to allow a user to individually adjust the securing device 110 to best fit around his or her head and/or helmet. While FIGS. 8 through 11 illustrate buckles 118 to adjust the length of the securing device 110, other means of adjusting the securing device 110 includes the use of elastic materials and weaves of fabrics, hook and loop fasteners, snaps, strings for tying, and other methods known in the art.

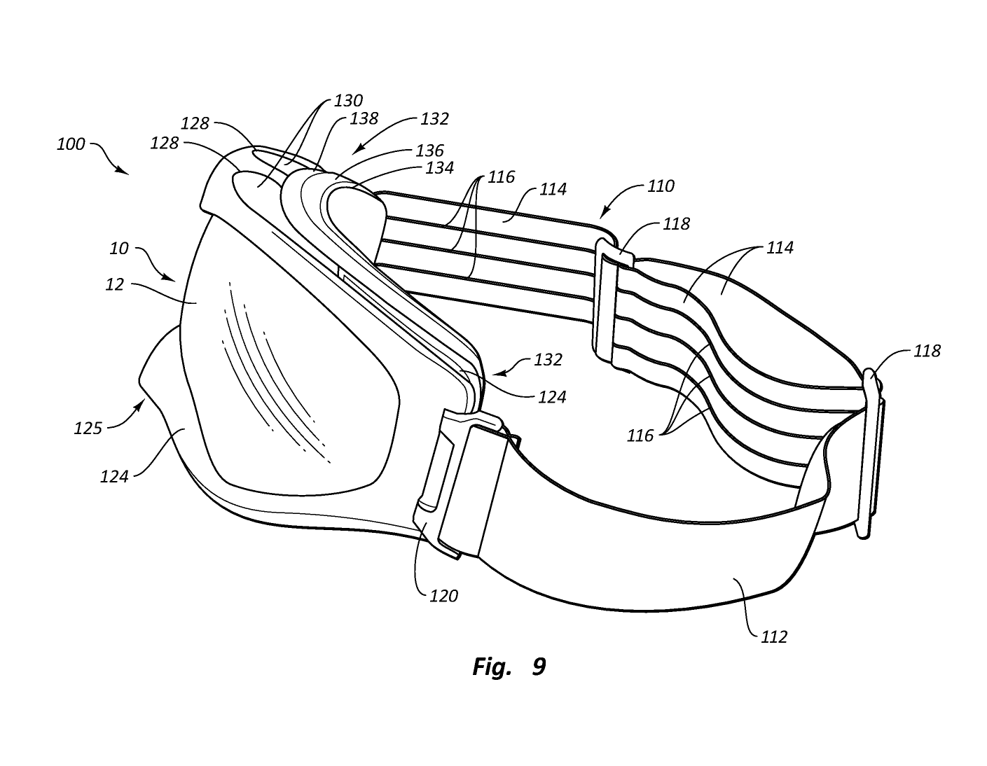

The upper frame vents 128 and upper frame vent foam 130 as discussed above are best illustrated in FIGS. 9 and 10. Similarly, lower frame vents 129 and lower frame vent foam 131, also previously discussed, are best illustrated in FIG. 11. The porous upper frame vent foam 130 and lower frame vent foam 131 allow the venting air 50 to escape from the interior space 140 of the frame 124 while minimizing or preventing snow, particulate matter, and other environmental irritants from entering the interior space 140. The upper and lower frame vent foam 130, 131 can be bonded to the frame 124 with various bonding agents as known in the art and as discussed above.

The frame 100 optionally includes sealing foam 132 that helps create a seal between the frame 124 and a user's face. Sealing foam 132 provides a flexible and generally weather proof seal about the user's face and better adjusts, with a flexible frame 124 and lens 110, to the shape of different users' faces.

The sealing foam 132 optionally comprises a plurality of different foams and other materials instead of just a single foam. For example, an embodiment of the sealing 132 illustrated in FIGS. 8 through 11 includes an outer fleece layer 134, a middle foam layer 136, and an inner foam layer 138.

The outer fleece layer 134 provides warmth and comfort against a user's skin while wicking moisture away from the user's face.

The middle foam layer 136 is bonded to the outer fleece layer 134 with bonding agents, such as adhesives, glues, chemical fixants, and the like. The middle foam layer 136 is a relatively more porous layer than the outer fleece layer 134. The middle foam layer 136 helps to quickly wick and absorb moisture from the outer fleece layer 134 and more quickly dissipate the moisture to the ambient air than would the outer fleece layer 134. Removing the moisture from proximate the user's face improves the user's comfort.

The third frame, or inner, foam layer 138 is bonded to the middle foam layer 136 and the frame 124 with bonding agents as discussed above. The frame foam layer 138 is a denser, i.e., less porous, foam than the middle foam layer 136. The denser frame foam layer 138 provides increased stability and better maintains its shape, thereby improving its ability to adhere to the frame 124 as the frame 124 flexes.

While the embodiments illustrated in FIGS. 2 through 11 relate to a goggle particularly adapted for outdoor use such as in skiing and snowboarding, other embodiments include safety glasses and goggles, visors for helmets, and other similar uses fall within the scope of the disclosure.

Methods of manufacturing embodiments of the invention are also disclosed herein. The method includes providing a first layer and a second layer of flat, transparent thermoplastic, such as polycarbonate. A first, polarized film is provided also provided. A chemical fixant, such as a bonding agent described above, is applied to at least part of one of the first layer and the polarized film. The polarized film is applied to the first layer such that polarized film adheres to the first layer. Of course it will be understood that the polarized film can be adhered to the second layer alternatively. A photochromatic film is also provided. A bonding agent is applied to at least part of one of the polarized film and the photochromatic film. The photochromatic film then is adhered to the polarized film. Of course, it will be understood that the photochromatic film can be adhered to the second layer initially instead of the polarized film. A bonding agent is applied to at least of the part of the photochromatic film and the second layer, which are then adhered together to form the embodiment illustrated in FIG. 1 in cross-section. Other arrangements and orders of combining the first layer, the second layer, the polarized film and the photochromatic film fall within the scope of the disclosure to form the lens 10.

The initially flat lens 10 is then thermoformed in a mold of a selected shape under a selected temperature and pressure to provide a selected radius of curvature in at least one of the horizontal and the vertical axis as described above. The process of forming the radius of curvature from a flat lens 10 occurs without causing any wrinkling, crackling, lines, cracking, and other defects in the lens 10 and the polarized film 48 and the photochromatic film 46 within the tolerances of the manufacturing process.

A frame for securing and positioning the lens 10 proximate a user's eyes is also provided, the frame including a securing device to hold the frame and lens in position about the user's head. The frame provided optionally includes a flexible thermoplastic, such as polyurethane, formed in a mold.

The following embodiments and figures of a goggle that incorporates an inner frame and an outer frame includes any and all of the features, whether singly or in all possible combinations, as described above and illustrated in FIGS. 1-11.

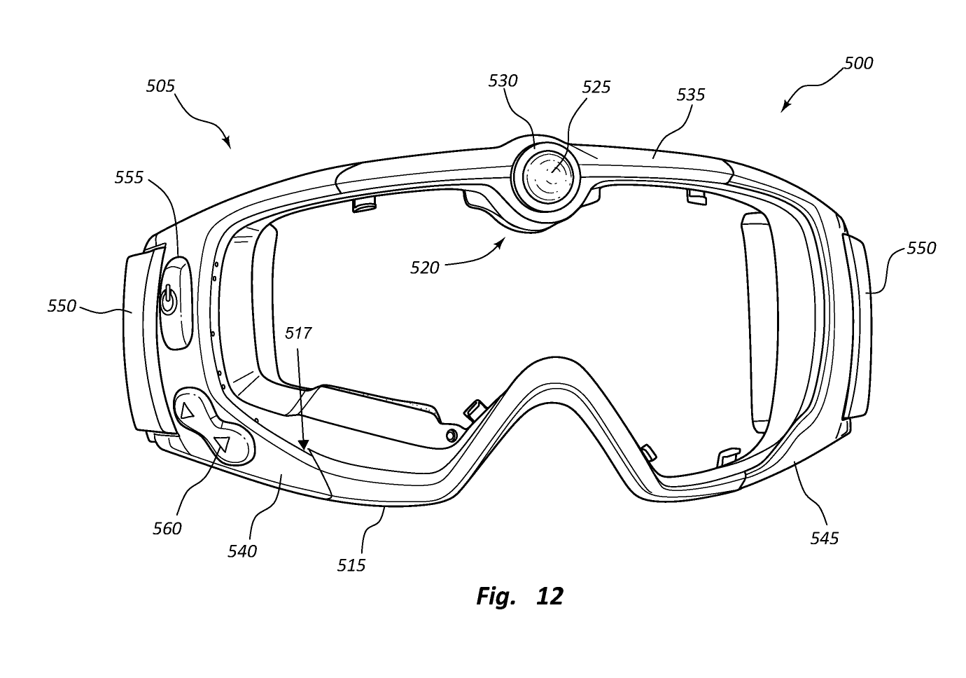

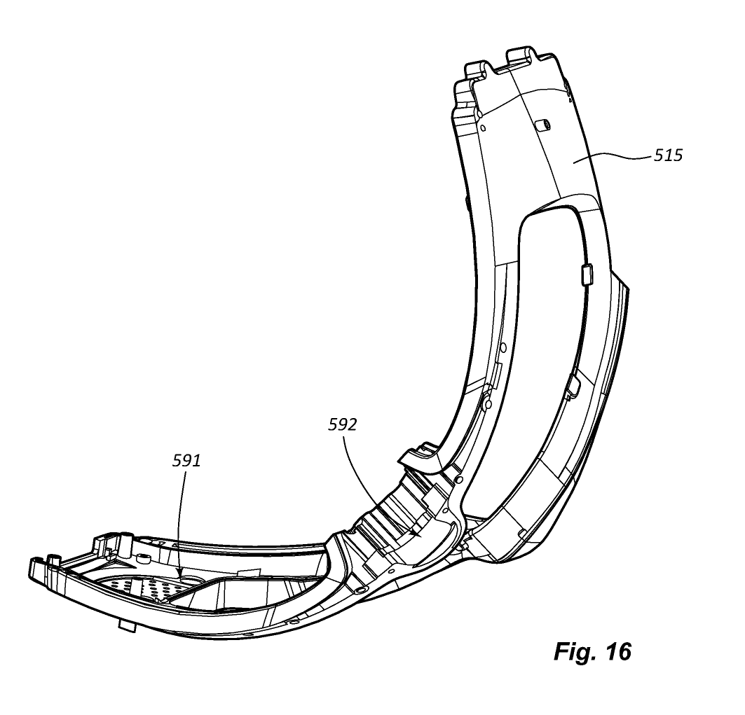

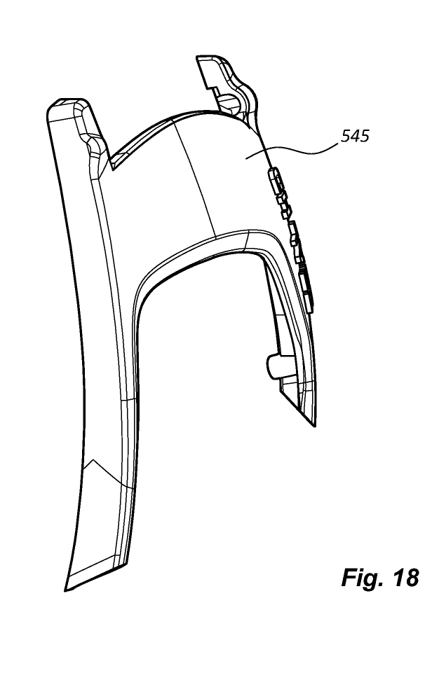

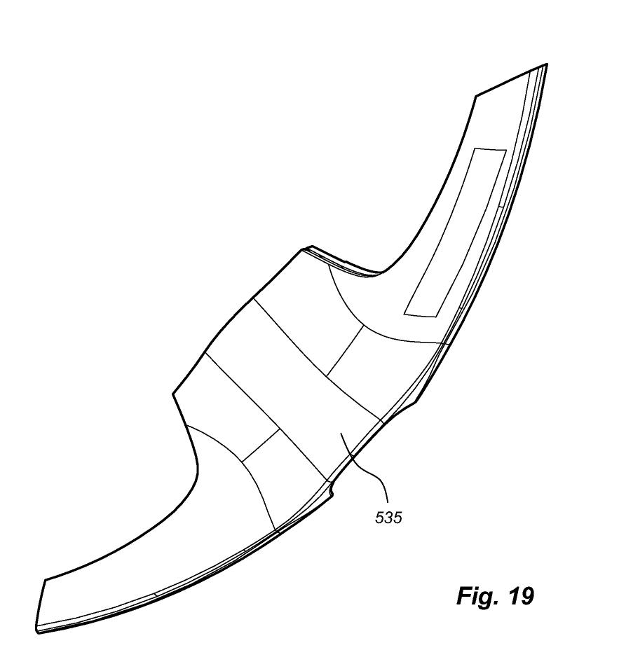

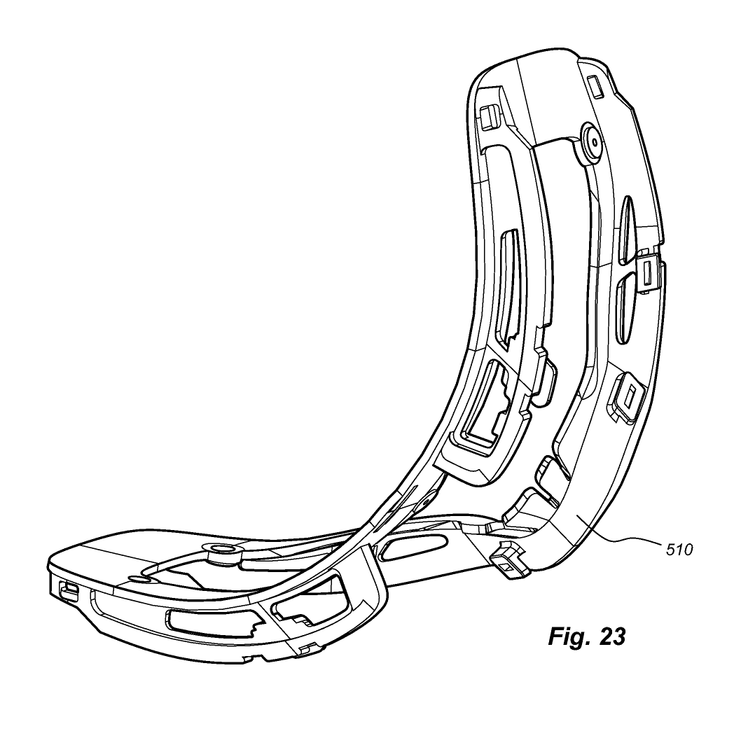

An embodiment of an all-weather goggle 500 having a frame 505 comprising a first or inner frame 510 and a second, or outer frame 515 is illustrated in FIGS. 12-25. FIGS. 12 through 14 illustrate front and rear perspective views of the goggle 500.

The frame 505 with a first frame 510 and a second frame 515 mix positive qualities of protection, mounting points and physical support for any optional electronics, and comfort. In some embodiments, an all-weather goggle 500 may simply be provided with an inner frame 510 and an outer frame 515 without any electronics.

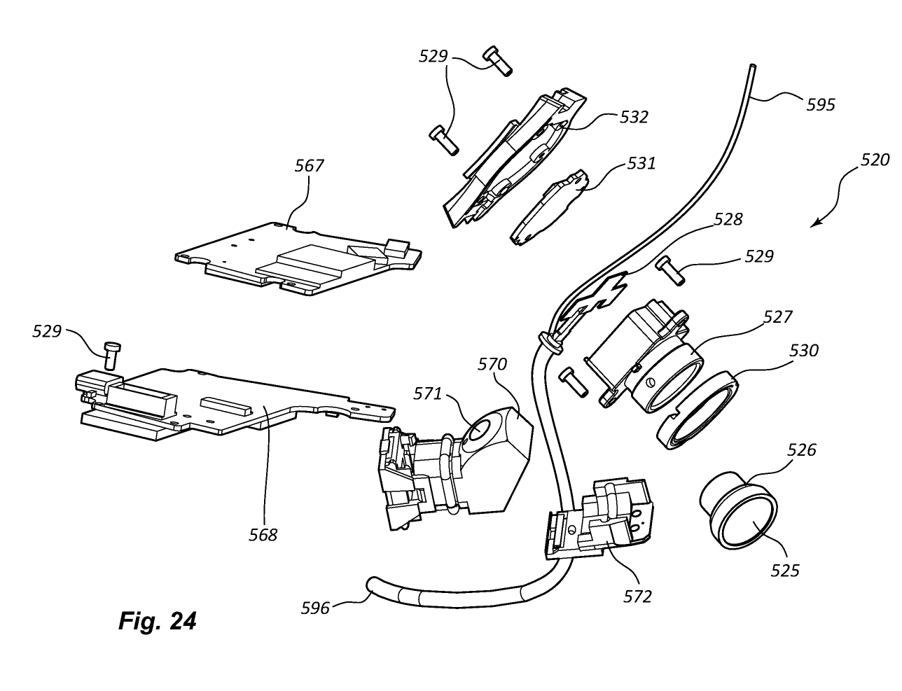

As illustrated, the goggle 500 optionally also includes a digital camera 520 capable of still and video pictures. The digital camera 520 and associated components, as illustrated in exploded view in FIG. 24, includes a lens 525, a lens housing 526, a lens ring 530, a lens mount 527, a semiconductor chip or imaging sensor 528 to capture an image and convert it into digital data in a storable medium, such as a charged coupled device, an imaging board 531, and a rear housing 532. Screws 529, or other attachment devices, such as bolts, adhesives, rivets, and the like, couple the lens mount 527 and, consequently the digital camera 520, to the second frame.

In those embodiments that include a wireless or wi-fi connection, a user may connect and interact with the electronics 567, 568, viewer package 570 (FIG. 24), digital storage, global positioning device, and digital camera 520 via a smart phone. Such interaction allows a user to connect to digital content on the all-weather goggle and allows the user to share the data through wireless communications, such as cellular, wi-fi, and social networking sites. In addition, the smart phone can operate to adjust various settings for the digital camera 520, act as a digital viewfinder and/or viewing screen for setting up, taking, and reviewing pictures and videos with the digital camera 520.

Figure illustrates a top perspective view of the outer frame 515 and an electronics recess 591 for at least partly receiving the electronic board or boards 567 and 568 discussed above and illustrated in FIG. 24. The electronic boards 567 and 568 can be mounted to the outer frame 515 via screws 529, adhesives, and other known ways of securing them to the frame 515.

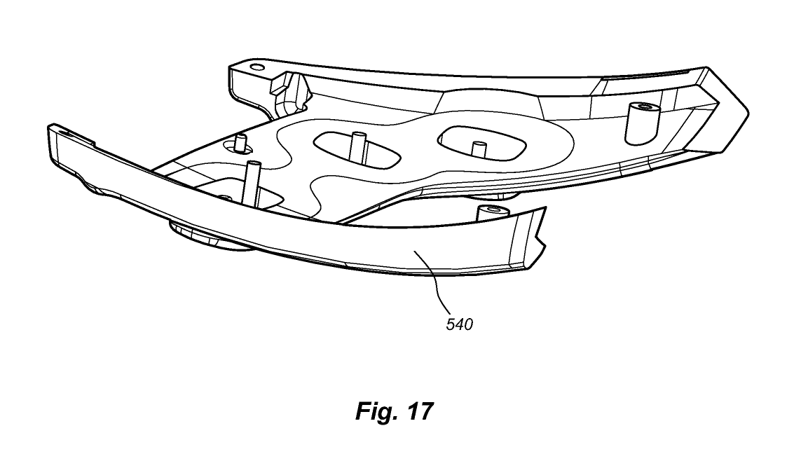

FIGS. 12, 13 and 17 through 16 illustrate covers of various types that couple to the outer frame 515. These covers include a top cover 535 (FIGS. 12, 13, and 19), right front cover 540 (FIG. 12 and FIG. 17 in an inside perspective view), and left front cover 545 (FIGS. 12, 13, and 18). These covers provide for a finish look and also, in some instances, partly or fully enclose one or more components, including at least one of a camera lens, a digital device to receive and store an image, a digital storage device, a view screen, an electronic board, a global positioning device, a digital communication device, such as wi-fi radio communications (send and receive) and hardwire connections (universal serial bus ports, for example), and a power source.

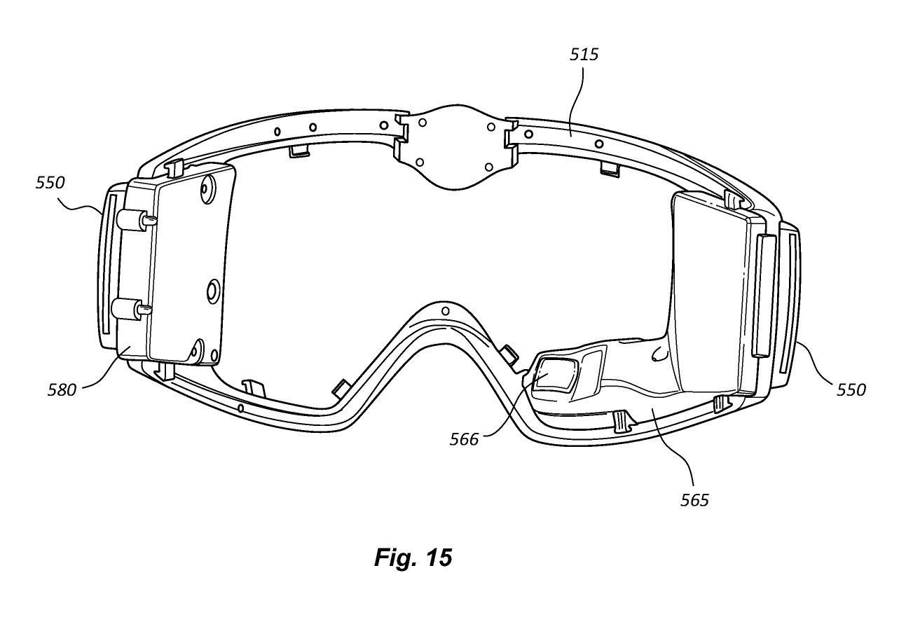

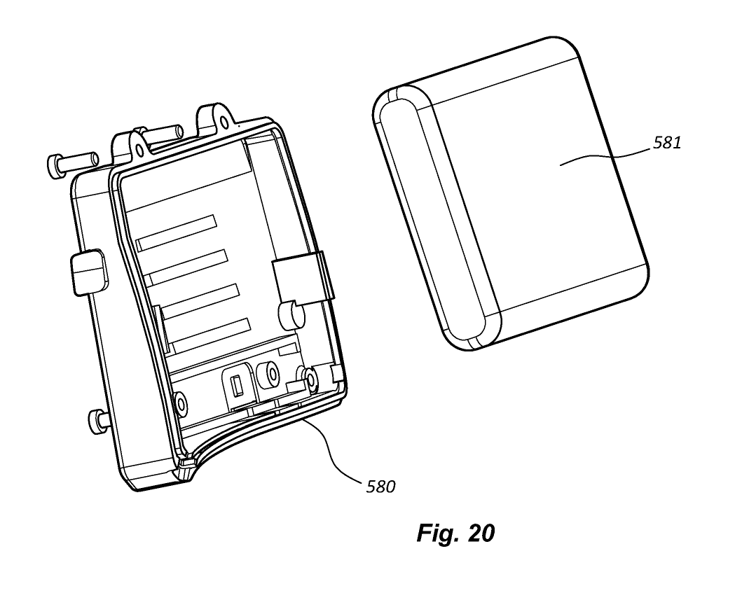

FIGS. 15 and 20 illustrate a left rear cover 580 that partially encloses a power source 581, such as a rechargeable battery.

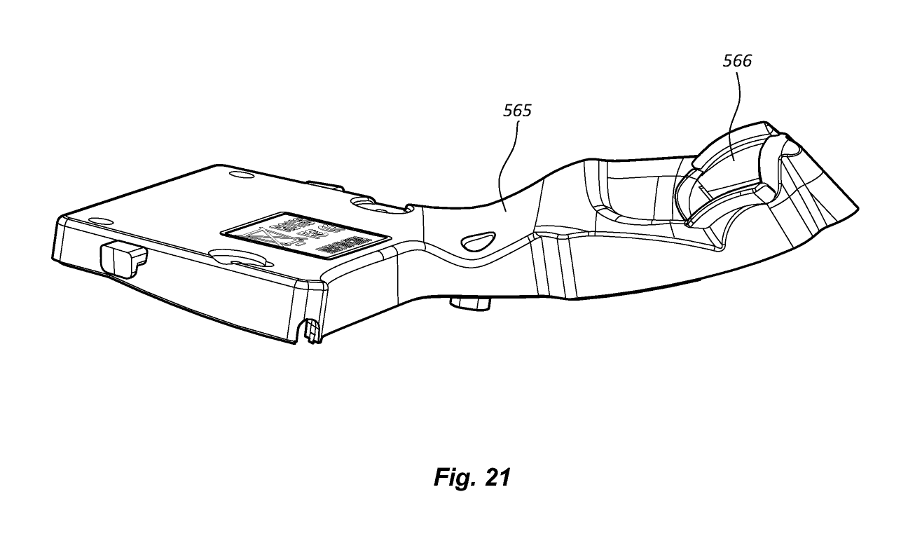

FIGS. 15 and 21 illustrate a cover 565 for the electronics 567, 568 and viewing screen 566. The viewing screen 566 covers the viewer package 570, which includes a view panel 571, such as a liquid crystal display (LCD) and a viewer package mount 572, as illustrated in FIG. 24.

Returning to FIGS. 12 through 14, brackets 550 provide an attachment location for the securing device described above. Buttons allow a user to control the electronics, including a power button 555 and a multifunction button 560, which may include two or more buttons and/or functions by pressing different portions of the button. As illustrated, the three buttons (555 and two-part multifunction button 560) perform all desired actions related to the aforementioned electronics, camera, and viewing device, which simplifies the user interface. Large buttons ease operation and allow easy identification and use through heavy gloves.

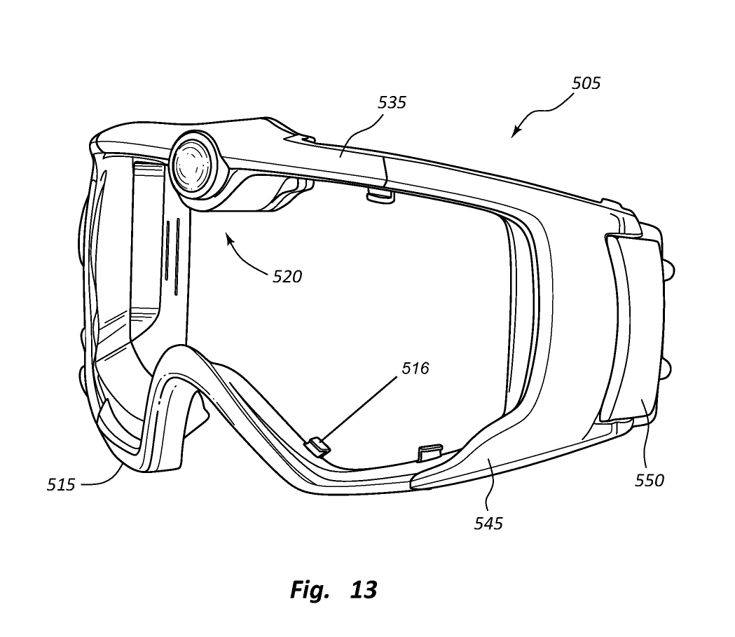

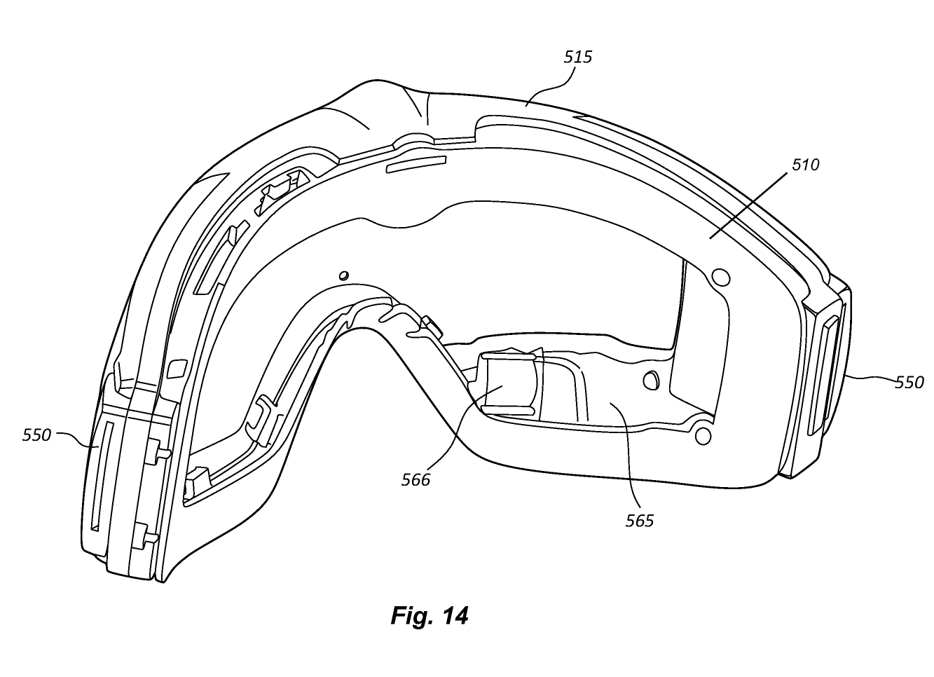

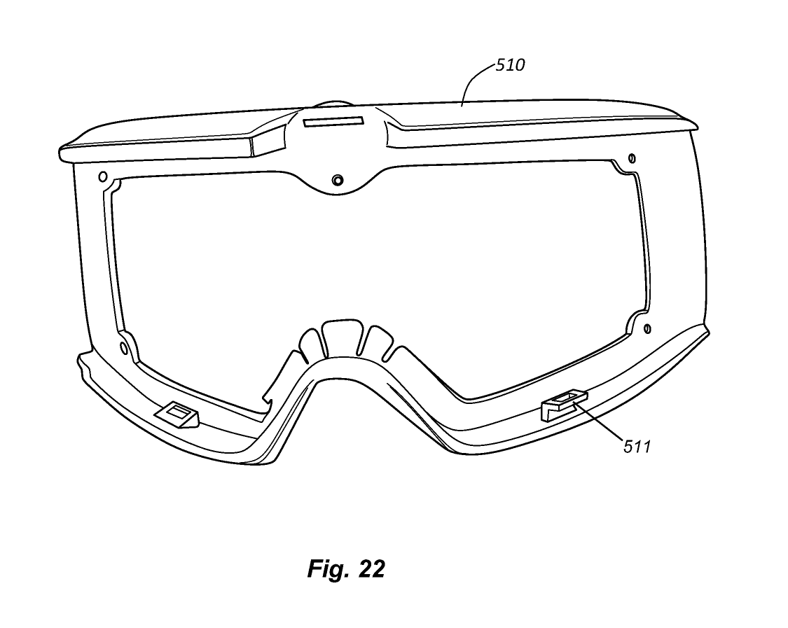

The first or inner frame 510 is illustrated in FIGS. 14, 22 and 23. The second or outer frame 515 is coupled to the first frame 510 via various possible securing devices, similar to screws 529 illustrated in FIG. 24, adhesives, press-fit slots 511 (FIG. 22) and tabs 511 (FIG. 13), and other known ways of coupling structures together. The sealing foam 132 discussed above is attached to the inner frame 510 on the side closest to the user's face.

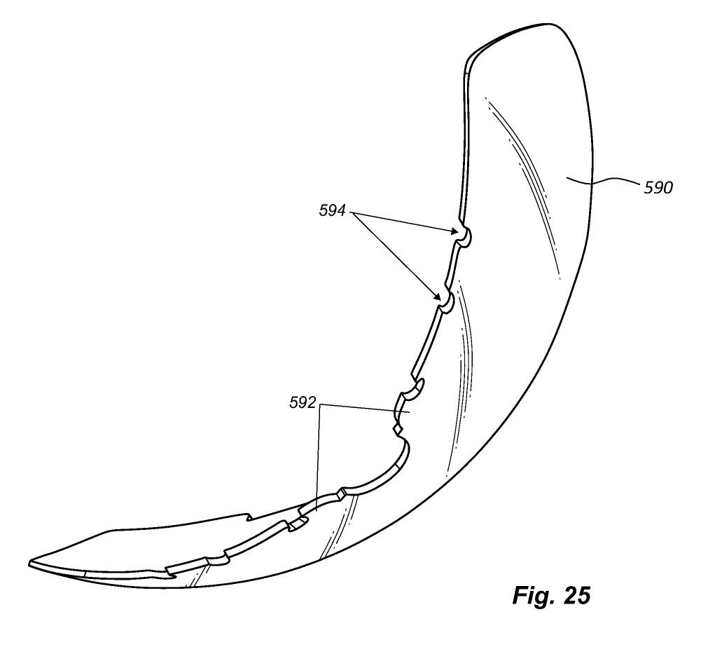

FIG. 25 illustrates a lens 590 with various tabs 592 and slots 594 for removable attachment to corresponding features, such as slot 517 (FIG. 12) in the second frame 515. The lens 590 may be of a type described above or any other type of lens used with all-weather goggles.

As briefly discussed above, FIG. 24 illustrates various types of electronics that might be attached to the outer frame 515. The cables 595 and 596 provide electrical connection between the power source 581 and the electronics boards 567, 568, the digital camera 520, and the viewing package 570. In addition to electrical current or power, the cables 595, 596 optionally include a signal path for digital communications so that digital data and/or operating instructions may be transmitted to and from the various components.

Returning to the discussion of the attributes of the inner frame 510 and the outer frame 515, the inner frame 510 conforms to the contours of the user's face and serves as an attachment point for the sealing foam described above. The inner frame 510 should provide flexibility, even under cold weather conditions. A first material from which the inner frame 510 includes a first elasticity as a material property. In various embodiments, the inner frame 510 is made from a thermoplastic polyurethane.

The outer hard 515 frame provides a high level of impact resistance and secures the lens 590 to the frame 515. The outer frame 515 is made of a second material that has an elasticity as a material property that is less than the elasticity of the first material from which the inner frame 510 is made. This provides the outer frame 515 with relatively greater rigidity than the first frame 510. The outer frame 515 is therefore sufficiently rigid to support and to protect any electronics connected to it. In addition, the outer frame 515 maintains the arcuate shape of the lens 590 when the user wear's the goggle 500 and when the goggle 500 is not worn. Alternatively, the elasticity of the outer frame 515 is sufficiently rigid or provides sufficient rigidity to hold at least one of the major axis 36 and the minor axis 30 substantially constant when said goggle 500 is worn by the user and when the goggle 500 is not worn by the user.

In various embodiments, the inner frame 510 is made from a thermoplastic polyamide. In various embodiments, the inner frame 510 is made from thermoplastics in the Grilamid® family provided by EMS-Grivory of Switzerland. In various embodiments, the inner frame 510 is made from Grilamid® TR90 provided by EMS-Grivory of Switzerland.

The outer frame 515 at least partially encloses or encapsulates any or all of the various electronic components described above, providing the electronic components with water-resistance. Thus, appropriate seals, such as O-rings, gaskets, and the like may be included between the first frame 510 and the second frame 515 and the various other covers to provide and/or enhance the water-resistance provided to the various electronic components. In some embodiments, the outer frame 515 fully encloses or encapsulates any or all of the various electronic components described above, providing the electronic components with water-resistance.

The one or more present inventions, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure.

The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. An all-weather goggle to be worn about a user's head, said all weather goggle comprising:
a lens formed and sized to extend about the eyes to cover the eyes and eye area of a user, said lens having an arcuate shape;
a frame configured for holding and positioning the lens proximate the eyes of the user, said frame including:
an inner frame for positioning against a user's face, said inner frame being made of a first material having a first elasticity;
an outer frame configured to receive and to hold said lens, said outer frame being coupled to said inner frame, said outer frame being made of a second material having a second elasticity that is less than said first elasticity;
a securing device associated with said frame for holding said frame proximate the eyes of a user.

2. The all-weather goggle of claim 1, wherein said second elasticity of said outer frame provides sufficient rigidity to maintain said arcuate shape of said lens when said goggle is worn by said user and when said goggle is not worn by said user.

3. The all-weather goggle of claim 1, wherein said lens further comprises at least one of a major axis extending generally from one side to the other side of the users head in alignment with the user's eyes and a minor axis extending from the forehead downward toward the user's cheeks, and wherein said second elasticity of said outer frame provides sufficient rigidity to hold the at least one of said major axis and said minor axis substantially constant when said goggle is worn by said user and when said goggle is not worn by said user.

4. The all-weather goggle of any of claim 1, wherein said inner frame is made of a thermoplastic polyurethane and said outer frame is made of a thermoplastic polyamide.

5. The all-weather goggle of claim 1, wherein said outer frame is configured to retain at least one of a camera lens, a digital device to capture an image, a digital storage device, a view screen, an electronic board, a global positioning device, and a power source.

6. The all-weather goggle of claim 1, wherein said outer frame is configured to at least partly enclose at least one of a camera lens, a digital device to capture an image, a digital storage device, a view screen, an electronic board, a global positioning device, and a power source.

7. The all-weather goggle of claim 1, wherein at least one cover is coupled to said outer frame so that said cover and said outer frame enclose at least one of a camera lens, a digital device to capture an image, a digital storage device, a view screen, an electronic board, a global positioning device, and a power source.

8. An all-weather goggle to be worn about a user's head, said all weather goggle comprising:
a lens formed and sized to extend about the eyes to cover the eyes and eye area of a user, said lens having an axis extending generally from one side to the other side of the users head in alignment with the user's eyes, said lens including at least one tab around a periphery of said lens;
a frame configured for holding and positioning the lens proximate the eyes of the user, said frame including:
an inner frame for positioning against a user's face, said inner frame being made of a first material;
an outer frame that includes at least one slot configured to receive said at least one tab of said lens, said outer frame holding said lens so that said axis remains substantially constant when said goggle is worn by said user and when said goggle is not worn by said user, said outer frame being coupled to said inner frame, said outer frame being made of a second material; and,
a securing device associated with said frame for holding said frame proximate the eyes of a user.

9. The all-weather goggle of claim 8, wherein said lens includes another axis extending from said user's forehead downward toward said user's cheeks and wherein said outer frame holding said lens so that said another axis remains substantially constant when said goggle is worn by said user and when said goggle is not worn by said user.

10. The all-weather goggle of any of claim 8, wherein said inner frame is made of a thermoplastic polyurethane and said outer frame is made of a thermoplastic polyamide.

11. The all-weather goggle of claim 8, wherein said outer frame is configured to retain at least one of a camera lens, a digital device to capture an image, a digital storage device, a view screen, an electronic board, a global positioning device, and a power source.

12. The all-weather goggle of claim 8, wherein said outer frame is configured to at least partly enclose at least one of a camera lens, a digital device to capture an image, a digital storage device, a view screen, an electronic board, a global positioning device, and a power source.

13. The all-weather goggle of claim 8, wherein at least one cover is coupled to said outer frame so that said cover and said outer frame enclose at least one of a camera lens, a digital device to capture an image, a digital storage device, a view screen, an electronic board, a global positioning device, and a power source.

14. An all-weather goggle comprising:
a frame including:
a first portion being made of a thermoplastic polyurethane;
a second portion coupled to said first portion, said second portion being made of a thermoplastic polyamide;
a lens removably received and held in said frame proximate the eyes of a user; and,
a securing device associated with said frame for holding said frame proximate the eyes of a user.

15. The all-weather goggle of claim 14, wherein said lens further comprises at least one of a major axis extending generally from one side to the other side of the users head in alignment with the user's eyes and a minor axis extending from the forehead downward toward the user's cheeks, and wherein said second portion is configured to hold the at least one of said major axis and said minor axis substantially constant when said goggle is worn by said user and when said goggle is not worn by said user.

16. The all-weather goggle of claim 14, wherein said lens is generally arcuate in shape and said second portion is configured to maintain said arcuate shape of said lens when said goggle is worn by said user and when said goggle is not worn by said user.

17. The all-weather goggle of claim 14, wherein said first portion comprises an inner frame for positioning against a user's face and wherein said second portion comprises an outer frame that includes at least one slot configured to receive at least one tab around a periphery of said lens.

18. The all-weather goggle of claim 14, wherein said second portion is configured to retain at least one of a camera lens, a digital device to capture an image, a digital storage device, a view screen, an electronic board, a global positioning device, and a power source.

19. The all-weather goggle of claim 14, wherein said outer frame is configured to at least partly enclose at least one of a camera lens, a digital device capture an image, a digital storage device, a view screen, an electronic board, a global positioning device, and a power source.

20. The all-weather goggle of claim 14, wherein at least one cover is coupled to said second portion frame so that said cover and said second portion enclose at least one of a camera lens, a digital device to capture an image, a digital storage device, a view screen, an electronic board, a global positioning device, and a power source.
